(19) United States
(12) Patent Application Publication
(10) Pub. No.: US 2008/0077053 A1
(43) Pub. Date: Mar. 27, 2008

(54) Method for Vaginal Skin Biomechanical Evaluation

(76) Inventors: Lee Brandon Epstein, Louisville, KY (US); Carol Ann Graham, Prospect, KY (US); Michael Howard Heit, Louisville, KY (US)

Correspondence Address: BARNES & THORNBURG LLP, P.O. BOX 2786, CHICAGO, IL, 60690-2786, US

(21) Appl. No.: 11/470,195

(22) Filed: Sep. 5, 2006

(51) Int. Cl. A61B 5/103

(52) U.S. Cl. 600/591

(57) ABSTRACT

A method and technique to measure vaginal skin biomechanical properties in vivo by applying a temporary deforming force to the vaginal skin while using sensors to monitor and record the vaginal skins response to the deforming force as well as how it responds after the deforming force is removed. By placing a female participant in the correct anatomical position and employing a vaginal biomechanical evaluation tool to temporarily distort and measure the vaginal skin an examiner is able to obtain measurements for biomechanical properties of the vaginal skin. These measurements may allow clinicians and medical researchers to understand a woman's vaginal tissue quality, response to treatment, and risk for future pelvic floor disorders more completely in order to take appropriate prophylactic or corrective action.

### FIELD OF THE INVENTION

The present invention relates to the field of biomechanical evaluation of living tissue and in particular an improved method of vaginal skin characterization and evaluation.

### BACKGROUND OF THE INVENTION

As the population of the United States continues to increase in age the prevalence of diseases of female pelvic floor dysfunction including urinary incontinence, anal incontinence, pelvic organ prolapse, pain with sexual intercourse (dyspareunia), post menopausal vaginal atrophy, and cervical incompetence will rise substantially.

Recent research has demonstrated that these diseases may be related to changes in the underlying biomechanical properties of vaginal tissue. Despite mounting histological, biochemical, and genetic evidence to support these claims there remains no way to directly measure biomechanical parameters of vaginal tissue in vivo (in living subjects) without skin biopsy or surgical excision of the vaginal tissue. Examples of biomechanical parameters include, but are not limited to, elasticity (the capacity of skin to regain its original form after a deforming force is removed), extensibility (the capacity of skin to be stretched), elastic modulus (stress required to produce a unit of strain), creep (time-dependent deformational change when skin is put under a persistent force), plasticity (the property of skin when it is permanently changed by a deforming force), and torsion (the capacity of skin to be deformed when exposed to a torsional force).

There is a need for non-invasive in vivo biomechanical evaluation techniques for the vagina to characterize and stratify a woman's risk for pelvic floor dysfunction in order to prevent it.

### SUMMARY OF THE INVENTION

It is an advantage of the present invention to provide non-invasive in vivo biomechanical evaluation techniques for the vagina to stratify a woman's risk for pelvic floor dysfunction such as urinary incontinence, anal incontinence, pelvic organ prolapse, pain with sexual intercourse (dyspareunia), post menopausal vaginal atrophy, and cervical incompetence.

It is a further advantage of the present invention to objectively measure the biomechanical effect medications have on vaginal skin and prove them effective.

It is a further advantage of the present invention to objectively measure the biomechanical effect corrective surgical treatments have on vaginal skin in order to compare them.

These and other advantages of the present invention are achieved by the present invention. In accordance with one embodiment, a new method and technique to measure vaginal skin biomechanical properties such as extensibility, elasticity, stiffness index, elastic modulus, creep, torsion, and plasticity is described.

In order to perform these vaginal biomechanical measurements a female participant is placed on a medical examination table in a dorsal lithotomy position. A vaginal speculum is inserted into the female participant's vagina and a suitable area of her vaginal skin (also referred to as the vaginal wall or vaginal mucosa) is chosen for further biomechanical evaluation. A biomechanical evaluation tool is then inserted into the female participants vagina and applied to the chosen area of the vaginal skin. The biomechanical evaluation tool applies a temporary deforming force to the vaginal skin. Sensors in and/or around the biomechanical evaluation tool are used to monitor and record the vaginal skins response to the initial deforming force as well as how it responds after the deforming force is removed. An examiner evaluates the female participants' vaginal biomechanical properties to understand more about the participants vaginal tissue quality, response to treatments, and risk for future pelvic floor disorders.

### BRIEF DESCRIPTION OF THE DRAWINGS

The present invention will be understood more fully from the detailed description given below and from the accompanying drawings of various embodiments of the invention, which, however, should not be taken to limit the invention to the specific embodiments, but are for explanation and understanding only.

FIG. 1 is a frontal view of a female participant in dorsal lithotomy position and prepared for vaginal biomechanical evaluation in accordance with an embodiment of the present invention;

FIG. 2 is a oblique and front view of a vaginal biomechanical evaluation tool in accordance with an embodiment of the present invention;

FIG. 3 is an illustration of a vaginal biomechanical evaluation tool against vaginal skin in the middle of a measurement cycle with vaginal skin lifted into the measurement cup in accordance with an embodiment of the present invention;

FIG. 4 is an illustration of a vaginal biomechanical evaluation tool against vaginal skin at the end of a measurement cycle after vaginal skin returns to its normal shape in accordance with an embodiment of the present invention;

FIG. 5 is a front view of a vagina with anatomical landmarks and ideal vaginal biomechanical measurement location identified in accordance with an embodiment of the present invention;

FIG. 6 is a front view of a female participant's vagina and perineum with the vaginal biomechanical evaluation tool in place for measurement in accordance with an embodiment of the present invention; and

FIG. 7 is a basic diagram of the components necessary to perform vaginal biomechanical evaluation including the vaginal biomechanical evaluation tool, the data processing center, and the computer for data analysis.

### DETAILED DESCRIPTION

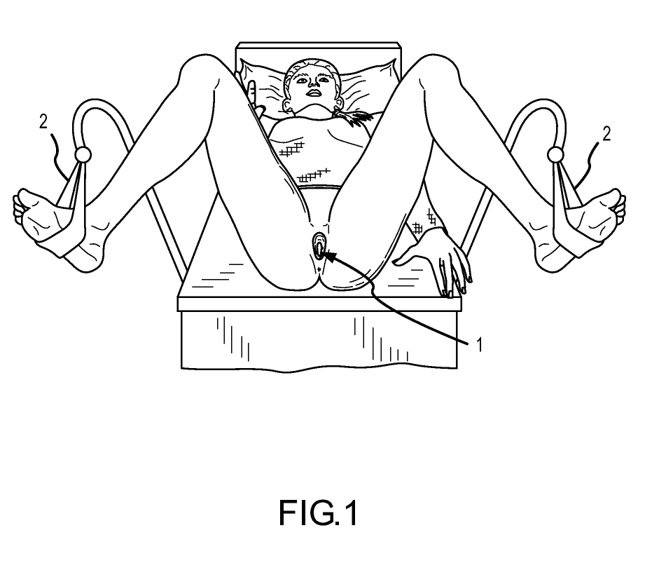

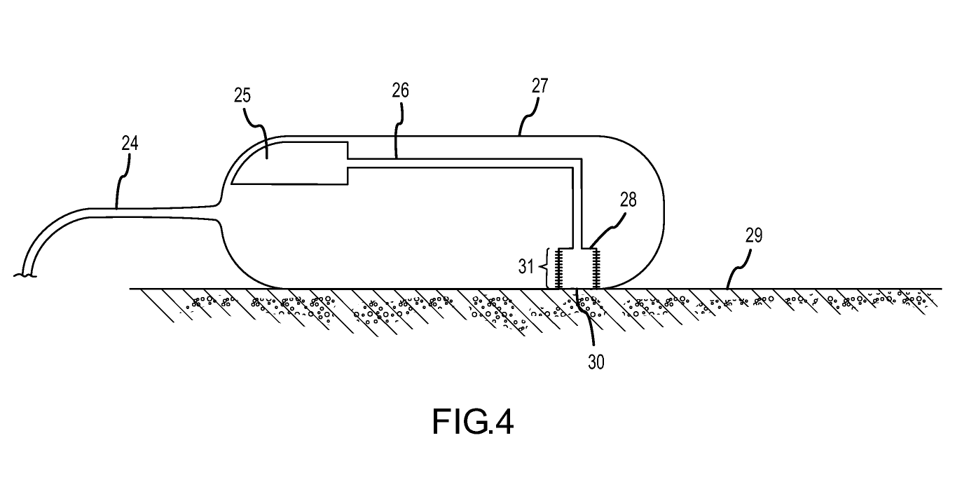

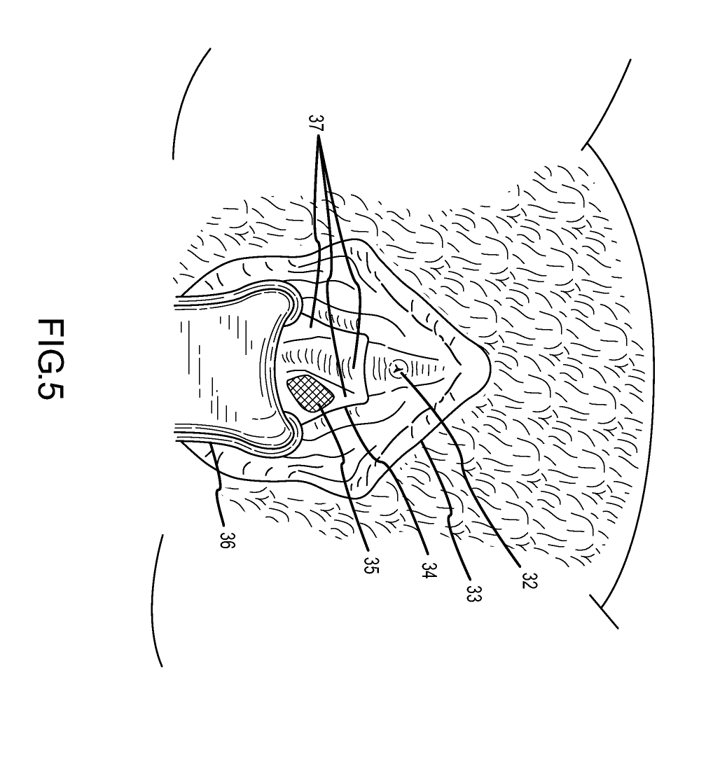

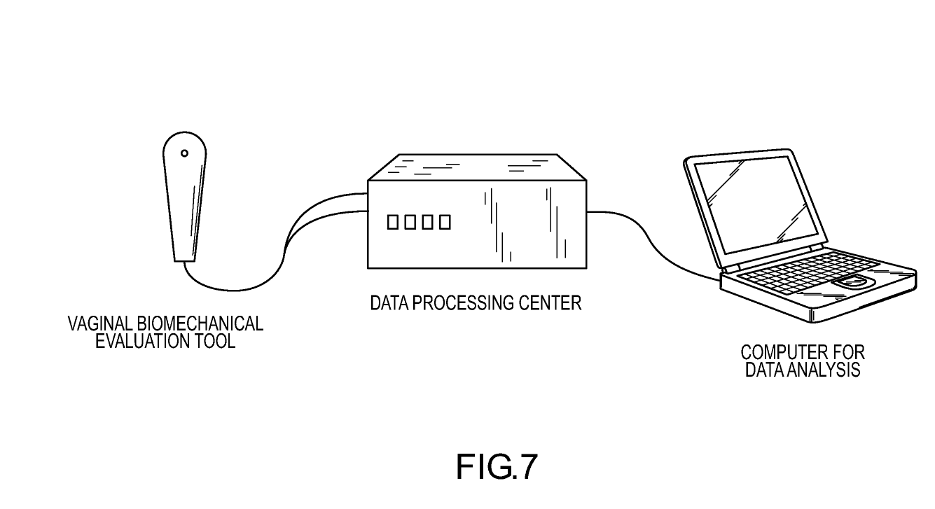

The present invention will now be described with reference to FIGS. 1-7 which in embodiments relate to a method for vaginal skin characterization and evaluation. It is understood that the present invention may be embodied in many different forms and achieved through the performance of many different steps and therefore should not be construed as being limited to the embodiments set forth herein. Rather, these embodiments are provided so that this disclosure will be thorough and complete and will fully convey the invention to those skilled in the art. Indeed, the invention is intended to cover alternatives, modifications and equivalents of these embodiments, which are included within the scope and spirit of the invention as defined by the appended claims. Furthermore, in the following detailed description of the present invention, numerous specific details are set forth in order to provide a thorough understanding of the present invention. However, it will be clear to those of ordinary skill in the art that the present invention may be practiced without such specific details.

The present invention is directed to the use of non-invasive in vivo biomechanical evaluation techniques for the vagina to evaluate, and stratify a woman's risk for, pelvic floor dysfunction such as pelvic organ prolapse, urinary incontinence, anal incontinence, dyspareunia, vaginal atrophy, and cervical incompetence. Biomechanical measurements taken by the present invention can also be used to evaluate the effects of medications, surgical therapies, and cosmetic agents on vaginal skin. Pelvic organ prolapse in particular has been demonstrated to be related to the extensibility of the vaginal skin. Pelvic organ prolapse can be defined as the hemiaion of uterus, small bowel, bladder, or rectum into the vaginal cavity and is the second leading indication for hysterectomy (removal of the uterus) in post-menopausal women in the United States. Despite many theories regarding the etiology of this disease surgical failure rates remain between 10-30% and there is currently no way to stratify a woman's risk for the disease. The inventors have demonstrated a linear relationship between the severity of pelvic organ prolapse and the extensibility of vaginal sidewall skin. The inventors hypothesize this relationship exists because vaginal skin extensibility is a surrogate marker for the quality of underlying connective tissues such as collagen and elastin. An elevated incidence of other diseases of the pelvic floor including urinary incontinence, anal incontinence, and cervical incompetence have also been related to deficiencies in these underlying connective tissues. These findings suggest that vaginal skin biomechanical properties including, but not limited to, extensibility and elasticity may be a measurable marker of a woman's risk for developing pelvic organ prolapse, urinary incontinence, anal incontinence, and cervical incompetence allowing for earlier intervention and lifestyle modification as a way to prevent disease.

In addition to its applicability for the evaluation of, and risk stratification for, pelvic floor dysfunction, the present invention achieves the objective measurement of biomechanical properties of vaginal skin to evaluate the effect of pharmaceutical agents or surgical treatments on the vagina. For example, there are many topical and systemic medications currently available for female post menopausal vaginal atrophy. Many of these products claim to increase the elasticity of the vagina and decrease sexual discomfort in these women. Up to this point only subjective measurements of vaginal elasticity have been available which are fraught with potential inaccuracies and sources of bias. Given that many of these patients are on a fixed income and the medications prescribed for vaginal atrophy can be quite expensive, an objective measurement of the biomechanical effect these medications have on vaginal skin and proof of their effectiveness is necessary. Furthermore, several surgical procedures directly involving the vagina have been hypothesized to decrease vaginal skin elasticity and lead to painful sexual intercourse but objective measurement techniques for vaginal skin elasticity have either been too invasive or too difficult to obtain to make them practical for studying these problems in large patient populations.

There are devices made for external skin biomechanical evaluation. These devices are commercially available. Examples of such devices include the Cutometer MPA 580 available from Courage and Khazaka Electronic GmbH in Cologne Germany, the DermaLab skin elasticity probe available from Cortex Technology in Hadsund Denmark, the Dermal Torque Meter available from Dia-Stron limited in Hampshire United Kingdom, and the Torsional ballistometer also available from Dia-Stron limited in Hampshire United Kingdom. However, none of these devices have been developed for vaginal applications nor is a method or technique to use these devices in the vagina available.

According to the present invention, to objectively measure the biomechanical properties of vaginal skin, some temporary deforming force is applied to the vaginal skin surface (either vacuum negative pressure suction, positive air pressure, rotational torsion, direct vaginal skin surface grasping with subsequent traction, pushing on the vaginal skin with an indenting object, stretching the vaginal skin surface parallel to the skin surface, or striking the skin with an indenting object) and sensors are used to evaluate how the skin responds to the initial deforming force as well as how it responds after the deforming force is removed. By closely monitoring the skin during these phases of testing (initial deforming force applied and reconstitution phase as the skin returns to its original form) it is possible to create a biomechanical description of the tested skin. This biomechanical description can include, but is not limited to, properties such as: elasticity (the capacity of skin to regain its original form after a deforming force is removed), extensibility (the capacity of skin to be stretched), elastic modulus (stress required to produce a unit of strain), creep (time-dependent deformational change when skin is put under a persistent force), plasticity (the property of skin when it is permanently changed by the deforming force), and torsion (the capacity of skin to be deformed when exposed to a torsional force).

In order to perform vaginal skin biomechanical evaluation in accordance with the present invention, an examiner requires the following equipment: a female participant with a patent vagina, a biomechanical evaluation tool with its associated accessories, a computer for further data analysis, a bivalve vaginal speculum, and sterile gauze.

Referring to FIG. 1, initially the female participant will be asked to undress from the waist down to expose the vaginal orifice 1 and to lie with her back down on a standard medical examination table in a dorsal lithotomy position. This is a common position used for gynecological evaluation which allows the female participant to comfortably support her legs in medical stirrups 2 while exposing the vaginal orifice for examination and testing. Because this is the same position used for many gynecological surgeries which require anesthesia it is possible to perform vaginal biomechanical testing while the participant is fully awake or while they are anesthetized.

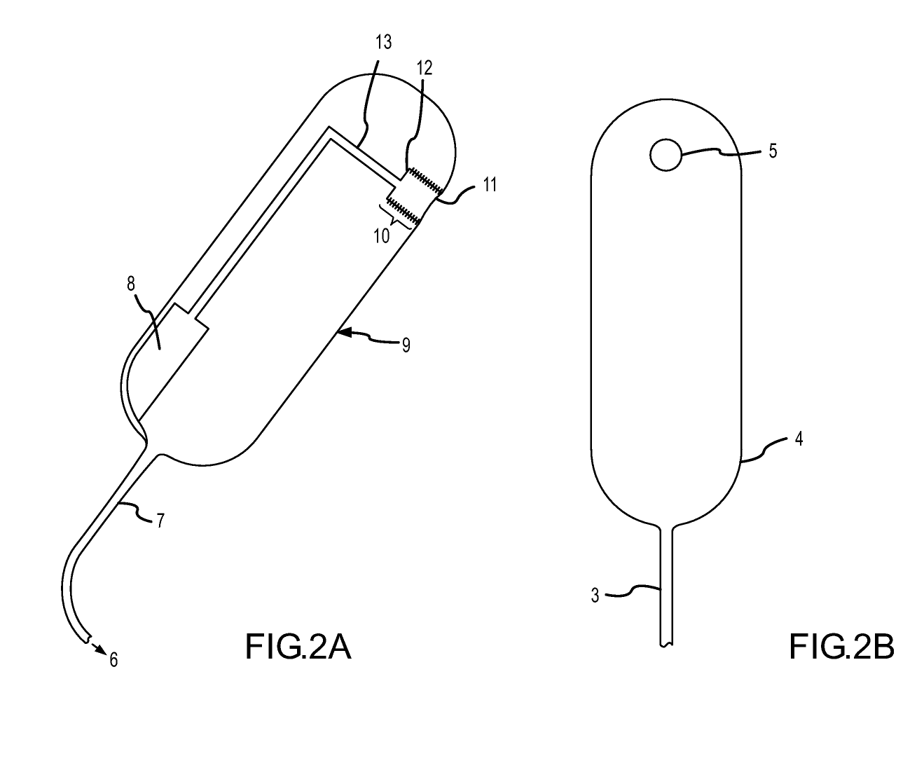

The next step for vaginal skin (also referred to as vaginal wall or vaginal mucosa) biomechanical evaluation is to prepare the vaginal biomechanical evaluation tool for use. Referring to FIG. 2, rod shaped device 9 is a biomechanical evaluation tool in accordance with one embodiment of the present invention. Rod shaped device 9 employs vacuum negative pressure to provide the temporary deforming force to the vaginal skin surface and infrared sensors 10 to monitor the resultant vaginal skin changes 21. Sensors other than infrared sensors such as laser based, temperature based, mechanical based, ultrasound based, ultrasonic based, or pressure based sensors may be employed. In addition, the biomechanical evaluation tool can be manufactured in many different shapes or sizes and the type of temporary deforming force as well as the method of monitoring the vaginal skin for changes may be different then those described in the preferred embodiment of this device. Measurement cup 12 should first be checked to ensure that no foreign bodies or detritus have obscured the infrared sensors 10. If present they should be removed using gauze pads only. At this point in preparation of the biomechanical evaluation tool the communication cord 3 should be attached to a data processing center and computer capable of managing the experimental protocol, data collection, and data analysis for the vaginal biomechanical evaluation tool 9. This computer may also be connected to the internet to facilitate data sharing/storage between different locations as well as remote site data analysis.

Referring to FIGS. 1, 2 and 5, the female participant should be in a comfortable dorsal lithotomy position with her vaginal orifice 1 even with the end of the examination table and should be ready for a vaginal speculum exam. A vaginal speculum is an instrument commonly used in gynecology to gently spread apart the walls of the vagina facilitating better visualization of the vaginal walls 37, cervix, and vaginal apex. Next, the vaginal speculum is introduced into the vaginal orifice per commonly accepted gynecological technique. The blades of the vaginal speculum are initially closed together and the speculum is inserted into the vagina at least 5-6 cm before the blades of the vaginal speculum are opened. Following this, the walls of the vagina 37 should be plainly in view to the examiner.

With the vaginal walls 37 in clear view to the examiner, a site on the vaginal skin should be identified and prepared for biomechanical evaluation. A point on one of the vaginal walls approximately 2 cm internal to the remnant of the hymenal ring is an ideal location for further consideration and examination 35. Once the area is identified it should be dried off and cleaned gently with sterile gauze pads to help facilitate vaginal testing with the vaginal biomechanical evaluation tool. After this the vaginal biomechanical evaluation tool 9 can be advanced through the blades of the vaginal speculum and rested against the vaginal skin with the measurement cup orifice 5 facing the vaginal skin 35. At this point the vaginal biomechanical evaluation tool may be secured to the vaginal skin using either double sided tape or manual pressure from the examiner. It is also conceivable that a location of vaginal skin could be chosen for evaluation, and the biomechanical evaluation tool could be placed, without being directly visualized.

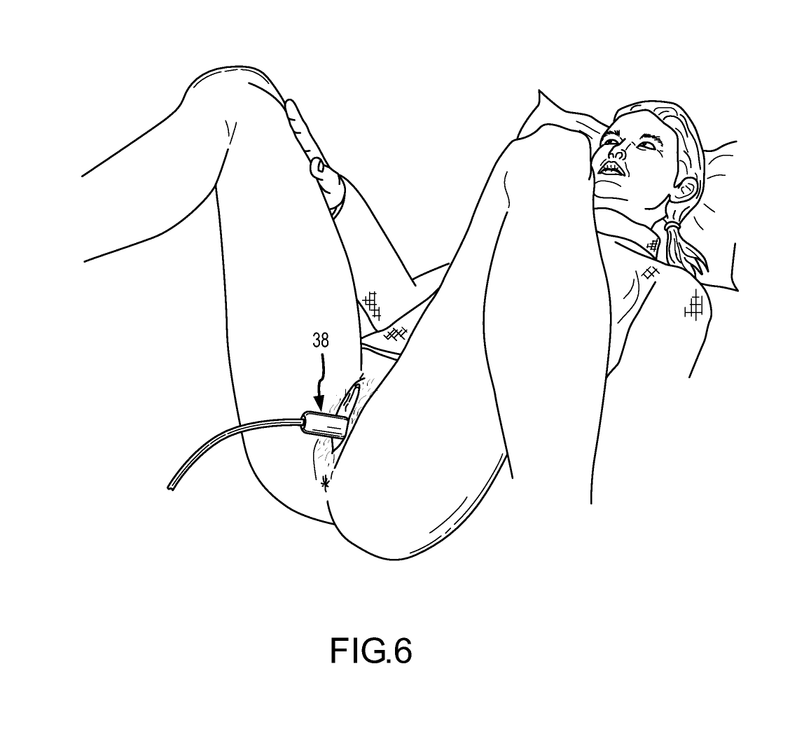

After the vaginal biomechanical evaluation tool is placed against the vaginal sidewall the vaginal speculum can be carefully removed from the vagina. Care should be taken to hold the vaginal biomechanical evaluation tool 9 in place against the vaginal skin while removing the vaginal speculum. When holding the vaginal biomechanical evaluation tool 9 against the vaginal skin the examiner should use uniform and gentle pressure. FIG. 6 illustrates what the examiner should expect to see after the vaginal speculum is removed.

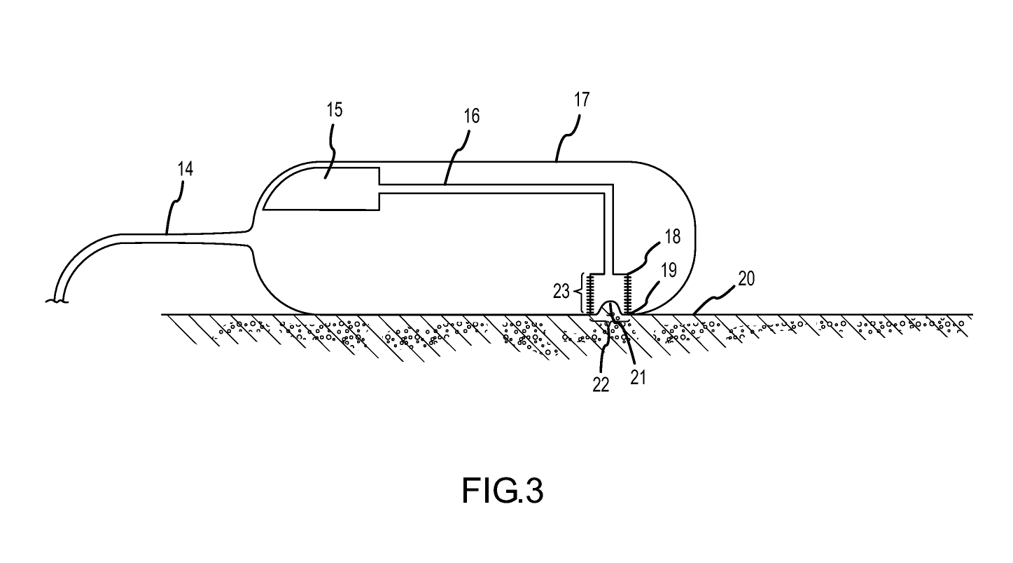

Once the vaginal biomechanical evaluation tool is in the correct position on the vaginal wall, data collection can begin. Referring to FIG. 3, the vaginal biomechanical evaluation tool uses multiple infrared sensors 10 and a vacuum pump 8 housed in the device itself to apply a distorting force on the vaginal wall surface 21. The face of the vaginal biomechanical evaluation tool with the measurement cup orifice 5 is placed against the vaginal wall surface 20 to be tested and a continuously increasing negative vacuum pressure is applied to the area of exposed vaginal wall 22 at the base of the measurement cup 19. This exposed area of vaginal wall surface will serve as the area to be tested 22.

As the negative vacuum pressure increases in strength the exposed skin 22 will be pulled into the measurement cup 12 past the different infrared sensors 10. As the vaginal skin passes each one of these infrared sensors 10, the computer records the time since vacuum force application began, as well as the negative vacuum pressure necessary to pull the vaginal skin to that sensor. The negative vacuum pressure continues to increase until the dome of exposed vaginal skin crosses the final infrared sensor beam 18 located 2.5 mm from the vaginal wall. The computer records the negative vacuum pressure, and time since vacuum force application needed to reach this high infrared sensor.

After the exposed vaginal skin crosses this final infrared sensor beam 18, the vacuum pressure is released and the vaginal wall is allowed to return to its original shape 30. The rate at which the vaginal skin returns back to its original shape and the time necessary for the vaginal skin to return back to its original shape will also be recorded by the computer. Because the area of vaginal skin exposed to vacuum pressure is constant and the distance between the infrared sensors is known, the pressure and time necessary to move the vaginal skin between these different infrared sensors, both while vacuum force is applied as well as after vacuum force is removed, can be used to calculate the biomechanical properties mentioned above including: elasticity, extensibility, elastic modulus, creep, and plasticity. A biomechanical measurement “cycle” will be referred to as starting from the time that vacuum force is applied to the vaginal skin and ending after the vacuum force is stopped and the vaginal skin returns back to its original shape. Measurement cycles can also be repeated successively to determine how the vaginal skin responds to such repeated stresses. Because of the laxity of vaginal skin, it is recommended that a relaxation period between measurement cycles is at least 10 seconds to allow adequate time for the vaginal skin to return to its original shape.

The vaginal biomechanical evaluation tool should be prepared for use as described previously and an experimental protocol is described here. In accordance to one embodiment of the present invention, continuously increasing negative vacuum pressure (suction), 10 second relaxation time between measurement cycles, and 2 cycles of measurement has proven effective. Measurement values for both time and negative pressure necessary to reach each of the infrared sensors will be recorded for later processing and biomechanical property calculation by the computer.

After 2 measurement cycles are completed and the data is collected, the vaginal biomechanical evaluation tool 9 can be removed from the female participant's vagina. The vaginal biomechanical evaluation tool should be removed from the female participant's vagina by simply pulling back on the handle portion 4 of the preferred embodiment of the tool. Otherwise, a vaginal speculum can be reintroduced into the female participant's vagina to visualize the vaginal walls and the vaginal biomechanical evaluation tool can be gently removed from the vagina under complete visualization. Once the vaginal biomechanical evaluation tool is removed from the female participant's vagina it should be inspected to ensure that it is fully intact and that no foreign objects or detritus have collected in the measurement cup 12 of the tool. The vaginal biomechanical evaluation tool should than be cleaned with either alcohol or germicidal wipes prior to use in another female participant.

By evaluating the biomechanical properties of the vaginal skin, as provided by the vaginal biomechanical evaluation tool, clinicians and medical scientists should be able to obtain a more complete qualitative and quantitative evaluation of vaginal skin. Such an evaluation may allow clinicians and medical researchers to understand a woman's vaginal tissue quality, response to treatments, and risk for future pelvic floor disorders more completely in order to take appropriate prophylactic or corrective action.

The inventors have demonstrated that increasing vaginal skin extensibility is associated with increasing pelvic organ prolapse severity in a dose dependant manner. Women with more significant pelvic organ prolapse (ie. more vaginal and pelvic tissue herniating down to, and out of, the vaginal opening) demonstrated increasing extensibility of the vaginal skin proportional to the amount of tissue herniation. Therefore, it is suggested that increased vaginal skin extensibility in women who do not currently manifest symptoms of pelvic organ prolapse may be an indicator of an increased risk for the disease in the future. Currently unaffected women who are at elevated risk of pelvic organ prolapse, as determined by vaginal skin biomechanical testing, could then be counseled to avoid activities, childbirth techniques, and lifestyle choices that further increase this risk. For example, women with highly extensible vaginal skin (ie. minimal vacuum negative pressure necessary to lift the vaginal skin past the infrared sensors corresponding with low negative pressure readings from the infrared sensors) may be at an increased risk for pelvic organ prolapse and would be counseled to avoid high risk activities such as vaginal child birth, jobs requiring heavy lifting, or hysterectomy without concurrent pelvic floor reconstruction. These women may also be at an increased risk for urinary and/or anal incontinence as well as cervical incompetence. Women with non-extensible vaginal skin (ie. maximal vacuum negative pressure necessary to lift the vaginal skin past the infrared sensors corresponding with high negative pressure readings from the infrared sensors) or non-elastic vaginal skin (ie. the vaginal skin is not able to regain its original shape, or takes a prolonged time to do so, after the vacuum negative pressure is removed corresponding with an elevated time necessary for the vaginal skin to retract below the lowest infrared sensor after the deforming force is removed) may be at an increased risk for dyspareunia. Furthermore, women with highly elastic vaginal skin (ie. the vaginal skin regains its original shape quickly after vacuum negative pressure is removed corresponding with a short time necessary for the vaginal skin to retract below the lowest infrared sensor after the deforming force is remove) may demonstrate a decreased risk for pelvic floor disorders such as urinary incontinence, anal incontinence, pelvic organ prolapse, pain with sexual intercourse (dyspareunia), vaginal atrophy, and cervical incompetence.

The drawings and description are not intended to represent the only form of the invention in regard to the details of construction and manner of operation. In fact, it will be evident to one skilled in the art that modifications and variations may be made without departing from the spirit and scope of the invention. Although specific terms have been employed, they are intended in a generic and descriptive sense only and not for the purpose of limitation, the scope of the invention being delineated in the claims that follow.

What is claimed is:

1. A method to measure vaginal skin biomechanical properties of a female subject comprising the steps of:
positioning the female subject so that the vagina is accessible;
inserting a biomechanical evaluation tool into the female subject's vagina;
applying a temporary deforming force to the vaginal skin with the biomechanical evaluation tool; and
detecting the vaginal skins' response to the deforming force.

2. The method of claim 1, wherein the step of detecting the vaginal skins' response to the deforming force is performed before the deforming force is removed.

3. The method of claim 1, wherein the step of detecting the vaginal skins' response to the deforming force is performed after the deforming force is removed.

4. The method of claim 1, wherein the step of detecting the vaginal skins' response to the deforming force is performed before and after the deforming force is removed.

5. The method of claim 1, wherein the force used to temporarily deform the vaginal skin is vacuum negative pressure.

6. The method of claim 1, wherein the force used to temporarily deform the vaginal skin is indentation or physical impact based force.

7. The method of claim 1, wherein the force used to temporarily deform the vaginal skin is one or more of: positive air pressure, rotational torsion, direct vaginal skin surface grasping with subsequent traction, or stretching the vaginal skin surface parallel to the skin surface.

8. The method of claim 1, wherein the vaginal wall is not inspected before biomechanical measurements are taken.

9. The method of claim 1, wherein the measure of vaginal skin biomechanical properties are used to evaluate a woman's risk for pelvic floor disorders such as urinary incontinence, anal incontinence, dyspareunia, pelvic organ prolapse, and vaginal atrophy.

10. The method in claim 1. wherein the measure of vaginal skin biomechanical properties are used to evaluate a woman's risk for cervical incompetence.

11. The method of claim 1, wherein the vaginal skin biomechanical properties are used to evaluate a woman's response to hormonal, medical, or surgical treatment.

12. The method of claim 1, wherein vaginal biomechanical properties are used as a surrogate measurement for underlying vaginal connective tissue, such as collagen and elastin, quality and strength.

13. The method of claim 1, wherein the deforming force is maintained at a constant for at least a period of time while applied to the vaginal skin.

14. The method of claim 1, wherein the deforming force is increased for at least a period of time while applied to vaginal skin.

15. The method of claim 1, wherein the deforming force is decreased for at least a period of time while applied to vaginal skin.

16. The method of claim 1, wherein the deforming force is increased and decreased for at least a period of time while applied to vaginal skin.

17. The method of claim 1, wherein the vaginal biomechanical evaluation tool employs infrared sensors to measure the skins' response to a deforming force.

18. The method of claim 1, wherein the vaginal biomechanical evaluation tool employs laser based sensors, temperature based sensors, mechanical based sensors, ultrasound based sensors, ultrasonic based sensors, or pressure based sensors to measure the vaginal skins' response to a deforming force.

19. The method of claim 1, wherein the female subject is not in the dorsal lithotomy position.

20. The method of claim 1, wherein some material other than sterile gauze is used to dry the vaginal skin.

21. The method of claim 1, further comprising the step of attaching the vaginal biomechanical evaluation tool to the vaginal skin.

22. The method of claim 21, wherein double-sided tape is used to attach the vaginal biomechanical evaluation tool to the vaginal skin.

23. The method of claim 1, wherein the female subject is either awake or anesthetized.

24. The method of claim 1, wherein 2 cycles of measurements are performed.

25. The method of claim 24, wherein the relaxation time between measurement cycles is other than 10 seconds.

26. The method of claim 1, wherein greater than 2 cycles of measurements are performed.

27. The method of claim 1, wherein animal vaginal tissue is used instead of human vaginal tissue.

28. The method of claim 1, wherein cadaveric vaginal tissue is used instead of living vaginal tissue.

29. The method of claim 1, wherein a vaginal speculum is not used at all or is inserted into the vagina to a depth of other than 5-6 cm.

30. The method of claim 1, wherein the vaginal biomechanical evaluation tool is placed anywhere along the vaginal skin, and/or on any vaginal wall (anterior, posterior, lateral, or apical).

31. The method of claim 1, further comprising the step of connecting a computer to the biomechanical evaluation tool such that the computer can receive data from the biomechanical evaluation tool.

32. The method of claim 31, further comprising the step of connecting the computer over a network such as the internet allowing for remote location data sharing, storage, or analysis.

33. A method to measure vaginal skin biomechanical properties of a female subject comprising the steps of:
positioning the female subject so that the vagina is accessible;
inspecting at least an area of vaginal skin;
drying at least the inspected area of vaginal skin;
inserting a biomechanical evaluation tool capable of applying a deforming force to vaginal skin into the female subject's vagina such that the biomechanical evaluation tool will apply a deforming force on or around the inspect area of vaginal skin;
attaching the biomechanical evaluation tool such that the biomechanical evaluation tool will apply a deforming force on or around the inspected area of vaginal skin;
applying a temporary deforming force on or around the inspected area of the vaginal skin with the biomechanical evaluation tool; and
detecting the vaginal skins' response to the deforming force.

34. The method of claim 33, wherein the step of detecting the vaginal skins' response to the deforming force is performed before the deforming force is removed.

35. The method of claim 33, wherein the step of detecting the vaginal skins' response to the deforming force is performed after the deforming force is removed.

36. The method of claim 33, wherein the step of detecting the vaginal skins' response to the deforming force is performed before and after the deforming force is removed.

37. The method of claim 33, wherein the force used to temporarily deform the vaginal skin is vacuum negative pressure.

38. The method of claim 33, wherein the force used to temporarily deform the vaginal skin is indentation or physical impact based force.

39. The method of claim 33, wherein the force used to temporarily deform the vaginal skin is one or more of: positive air pressure, rotational torsion, direct vaginal skin surface grasping with subsequent traction, or stretching the vaginal skin surface parallel to the skin surface.

40. The method of claim 33, wherein the measure of vaginal skin biomechanical properties are used to evaluate a woman's risk for pelvic floor disorders such as urinary incontinence, anal incontinence, dyspareunia, pelvic organ prolapse, and vaginal atrophy.

41. The method of claim 33, wherein the measure of vaginal skin biomechanical properties are used to evaluate a woman's risk for cervical incompetence.

42. The method of claim 33, wherein the vaginal skin biomechanical properties are used to evaluate a woman's response to hormonal, medical, or surgical treatment.

43. The method of claim 33, wherein vaginal biomechanical properties are used as a surrogate measurement for underlying vaginal connective tissue, such as collagen and elastin, quality and strength.

44. The method of claim 33, wherein the deforming force is maintained at a constant for at least a period of time while applied to the vaginal skin.

45. The method of claim 33, wherein the deforming force is increased for at least a period of time while applied to vaginal skin.

46. The method of claim 33, wherein the deforming force is decreased for at least a period of time while applied to vaginal skin.

47. The method of claim 33, wherein the deforming force is increased and decreased for at least a period of time while applied to vaginal skin.

48. The method of claim 33, wherein the vaginal biomechanical evaluation tool employs infrared sensors to measure the skins' response to a deforming force.

49. The method of claim 33, wherein the vaginal biomechanical evaluation tool employs laser based sensors, temperature based sensors, mechanical based sensors, ultrasound based sensors, ultrasonic based sensors, or pressure based sensors to measure the vaginal skins' response to a deforming force.

50. The method of claim 33, wherein the female subject is not in the dorsal lithotomy position.

51. The method of claim 33, wherein double-sided tape is used to attach the vaginal biomechanical evaluation tool to the vaginal skin.

52. The method of claim 33, wherein the female subject is either awake or anesthetized.

53. The method of claim 33, wherein 2 cycles of measurements are performed.

54. The method of claim 53, wherein the relaxation time between measurement cycles is other than 10 seconds.

55. The method of claim 33, wherein greater than 2 cycles of measurements are performed.

56. The method of claim 33, wherein animal vaginal tissue is used instead of human vaginal tissue.

57. The method of claim 33, wherein cadaveric vaginal tissue is used instead of living vaginal tissue.

58. The method of claim 33, wherein a vaginal speculum is not used at all or is inserted into the vagina to a depth of other than 5-6 cm.

59. The method of claim 33, further comprising the step of connecting a computer to the biomechanical evaluation tool such that the computer can receive data from the biomechanical evaluation tool.

60. The method of claim 59, further comprising the step of connecting the computer over a network such as the internet allowing for remote location data sharing, storage, or analysis.

61. The method of claim 33, wherein the vaginal biomechanical evaluation tool is placed anywhere along the vaginal skin, and/or on any vaginal wall (anterior, posterior, lateral, or apical).

62. The method of claim 33, wherein vaginal biomechanical properties are used to evaluate a woman's risk for pelvic floor disorders such as pelvic organ prolapse, urinary incontinence, anal incontinence, dyspareunia, and vaginal atrophy.
